(19) United States
(12) Patent Application Publication
(10) Pub. No.: US 2022/0183541 A1
(43) Pub. Date: Jun. 16, 2022

(54) SCOPE ENDCAP FOR NON-LINEAR PROCEDURES

(71) Applicant: Gyrus ACMI Inc., d/b/a Olympus Surgical Technologies America, Westborough, MA (US)

(72) Inventor: Anna K. Sczaniecka, Redmond, WA (US)

(21) Appl. No.: 17/553,169

(22) Filed: Dec. 16, 2021

Related U.S. Application Data
(60) Provisional application No. 63/126,196, filed on Dec. 16, 2020.

(51) Int. Cl. A61B 1/00; A61B 1/018

(52) U.S. Cl. CPC A61B 1/00098; A61B 1/00137; A61B 1/00096; A61B 1/018

(57) ABSTRACT

An apparatus for deflecting a medical tool passed through an endoscope. The apparatus includes a hollow proximal end having an internal diameter configured to provide a frictional fit to a distal end of an endoscope, a distal end having a first lumen extending from the hollow proximal end to a distal face, wherein the first lumen includes dimensions configured to receive a medical tool passed through a working channel of the endoscope, and a tube extending from the distal face at the lumen, wherein the tube has a precurved configuration configured to guide the medical tool received through the first lumen.

### PRIORITY CLAIM

This application claims the benefit of U.S. Provisional Application Ser. No. 63/126,196, filed Dec. 16, 2020, the contents of which are hereby incorporated by reference.

### DESCRIPTION OF THE RELATED ART

One of the challenges of peripheral sampling in the lungs is that often the sampling instrumentation tends to exit the distal end of the endoscope in a straight line. While this is isn't a problem when tumors are located directly in front of the endoscope or radial probe (concentric lesions), it is an issue when the tumors are located off to one side of the endoscope or radial probe (eccentric lesions). It is commonly accepted that eccentric lesions have a much lower diagnostic yield than concentric lesions. For this reason, some health care professionals (HCPs) wish for instrumentation that can be steered or directed off to one side.

### SUMMARY

An endcap for an endoscope is provided to more easily enable eccentric tissue sampling and/or drug deliver. An exemplary endcap attaches to the distal end of an endoscope. The endcap includes a feature for directing whatever tool is passed through the working channel of the endoscope to exit at an angle away from a longitudinal axis of the endoscope. Different types of endcaps may have exit ramps of varying lengths and angles. The endcap would be made of a clear material in order to not obstruct the scope camera or lights.

An exemplary apparatus includes a hollow proximal end having an internal diameter configured to provide a frictional fit to a distal end of an endoscope, a distal end having a first lumen extending from the hollow proximal end to a distal face, wherein the first lumen includes dimensions configured to receive a medical tool passed through a working channel of the endoscope, and a tube extending from the distal face at the first lumen, wherein the tube has a precurved configuration configured to guide the medical tool received through the first lumen. Alternatively, the tube could be bendable allowing for varying degrees of flexing.

In one aspect, at least one of the hollow proximal end, the distal end or the tube are transparent to a camera of the endoscope.

In another aspect, the distal end includes a second lumen that extends from the hollow proximal end to the distal face, the second lumen is configured to be aligned with a camera at a distal face of the endoscope when the working channel of the endoscope is aligned with the first lumen. The distal end includes a third lumen and a fourth lumen that extend from the hollow proximal end to the distal face, the third lumen and the fourth lumen are configured to be aligned with camera lights located at the distal face of the endoscope when the working channel of the endoscope is aligned with the first lumen.

In still another aspect, the tube incudes a stiffness value able to maintain the precurved configuration as the medical tool passes therethrough, such that as the medical tool exits the tube a longitudinal axis of the medical tool is at an acute angle relative to a longitudinal axis of the distal end of the endoscope.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates a prior art endoscope device.

FIG. 2 is an end view of a distal end of the endoscope device.

FIG. 3 is a side view of the distal end of the endoscope device of FIG. 2 and an endcap formed in accordance with one embodiment of the disclosed technology.

FIG. 4 is an isometric view of the endcap separated from the distal end of the endoscope device.

FIG. 5 is an isometric view of the endcap attached to the distal end of the endoscope device.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

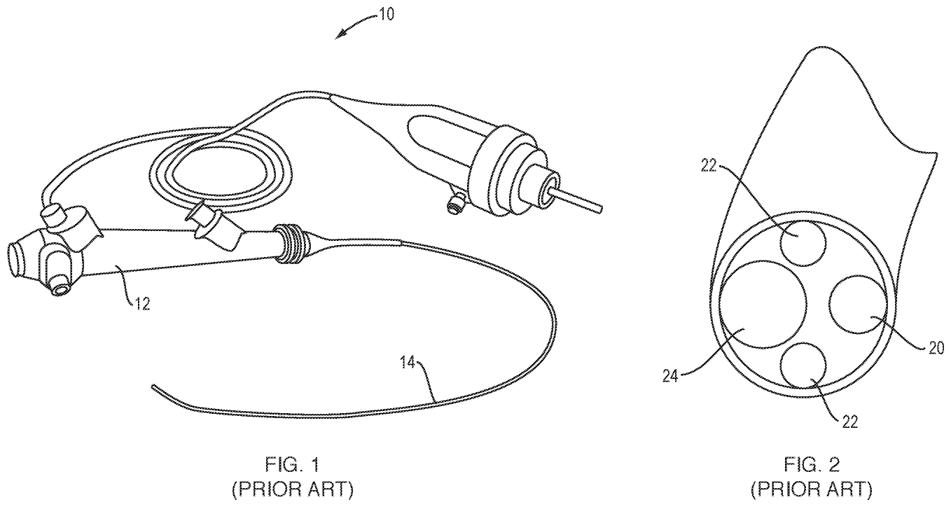

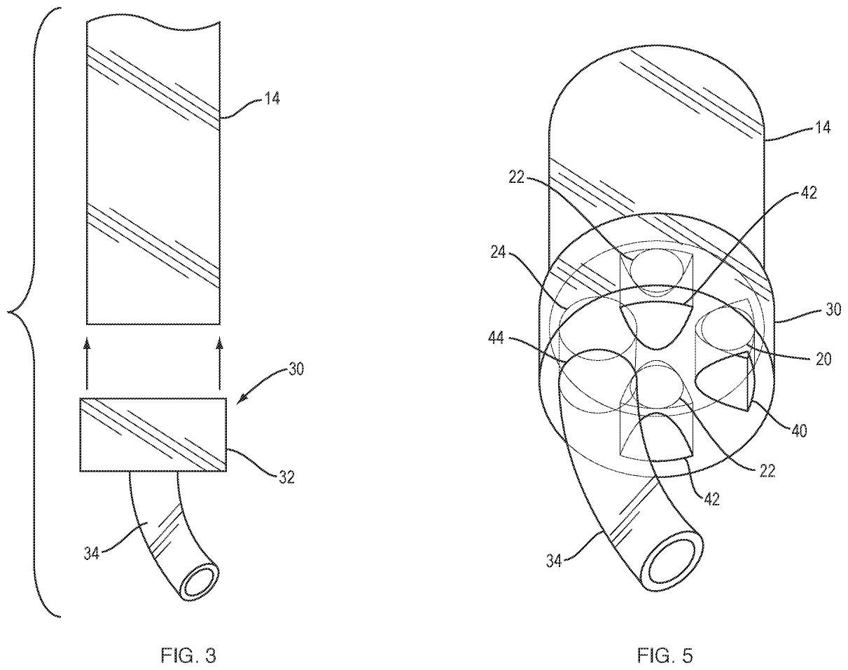

Referring now to FIGS. 1 and 2, an endoscope system 10 includes a endoscope 12 with an insertion tube 14. At the distal end of the insertion tube 14 is an camera 20 and lights 22. The insertion tube 14 includes a working channel 24 for slidably receiving medical devices (e.g., forceps, needle, cytology brush, cryoprobe, etc.)

The endoscope 12 is connected to a display processor and display device (not shown). The display device presents images based on information received from the camera 20. A diagnostic endoscope (e.g., endoscope BF-TH190 produced by Olympus®) is an example of the endoscope 12.

FIGS. 3-5 shows an endcap 30 and the distal end of the insertion tube 14. The endcap 30 attaches to the distal end of the insertion tube 14. The endcap 30 is partially or totally transparent for allowing the camera 20 to see past/through the endcap 30 to the surrounding tissue. As such, the camera 20 can generate video of the medical tool passing out of the insertion tube 14 thru the endcap 30 and into target tissue.

The endcap 30 includes four lumens 40, 42, 44 that extend longitudinally from a proximal face to a distal face of the endcap 30. When the endcap 30 is mounted to the insertion tube 14 (as shown in FIG. 5), the first lumen 40 is positioned relative (i.e., aligned) to the camera 20, the lumens 42 are positioned relative to the lights 22 and the lumen 44 is positioned relative to the exit port of the working channel 24.

A curved channel 34 extends from the distal face of the endcap 30 from the lumen 44. In one embodiment, the curved channel 34 curves toward the first lumen 40. When a user inserts a medical tool into the working channel 24 of the insertion tube 14, the user is able to see on the display when the distal end of the medical tool passes into the curved channel 34 and out a distal end of the curved channel 34.

The curved channel 34 may have a variety of different dimensions. For example, the curved channel 34 may be formed with any of a number of different lengths, internal lumen diameters or radii of curvature.

In one embodiment, the endcap 30 is at least partially formed (e.g., molded, 3D printed, etc.) of a high-performance thermoplastic elastomer compound, such as EARNESTON™ manufactured by KURARAY™. The elastomer material allows for easy attachment and removal to the insertion tube 14 without causing damage, while allowing for a secure attachment. In this embodiment, a proximal end 32 of the endcap 30 is hollow. An inner diameter is sized to allow the proximal end 32 to be slid over the distal end of the insertion tube 14. The proximal end 32 frictionally fits to the insertion tube 14 with enough force to overcome longitudinal forces experienced during a medical procedure without causing damage to the insertion tube 14. The frictional force holding the endcap 30 in place may be overcome by an operator post procedure.

In one embodiment, the endcap 30 is biocompatible and disposable. Also markings may be added to the curved channel 34 to improve visibility or assess distance.

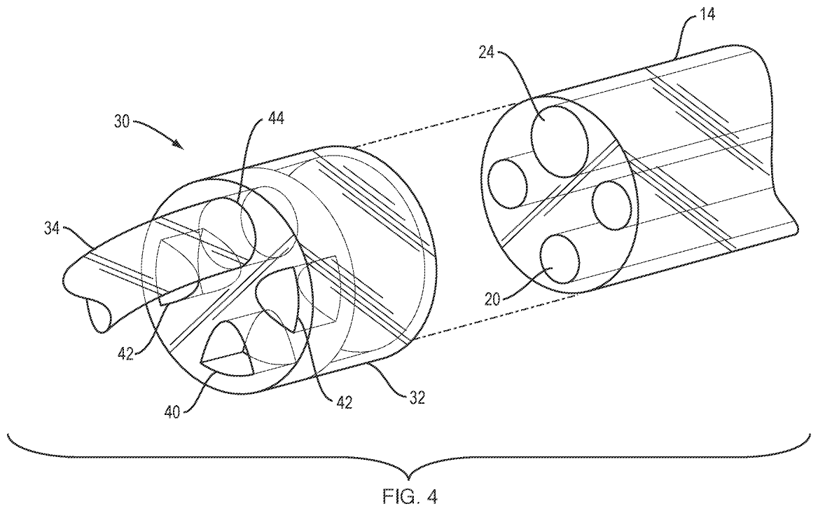

In one embodiment, alternative materials are used to facilitate the attachment of the endcap to the distal end of the endoscope. In this embodiment, the endcap does not need a hollow proximal section similar to that shown in FIG. 4. The endcap has an outer diameter that is approximately equivalent to the outer diameter of the insertion tube 14. An oversleeve (not shown) is then applied over a portion of the insertion tube 14 and a portion of the endcap for connecting the two components. The oversleeve may be shrunk, for example with heat, to provide friction fits to both the insertion tube 114 and the endcap.

Latex is one option, as are other materials, for securing the oversleeve to the distal tip. One example is EARNESTON™. The SmartBand® Multi-Band Ligation Kit, developed by OSTA in collaboration with the Southborough team/OEM supplier, was designed to endoscopically ligate internal hemorrhoids and esophageal varices at or above the gastroesophageal junction. These bands were designed to be anti-slip and latex free. These bands would also be gentle on the scope and allow for easy removal later on.

### EMBODIMENTS

A. An apparatus comprising: a hollow proximal end comprising an internal diameter configured to provide a frictional fit to a distal end of an endoscope; a distal end comprising a first lumen extending from the hollow proximal end to a distal face, wherein the first lumen comprises dimensions configured to receive a medical tool passed through a working channel of the endoscope; and a tube extending from the distal face at the first lumen, wherein the tube has a precurved configuration configured to guide the medical tool received through the first lumen.

B. The apparatus of A, wherein at least one of the hollow proximal end, the distal end or the tube are transparent to a camera of the endoscope.

C. The apparatus of A or B, wherein the distal end comprises a second lumen that extends from the hollow proximal end to the distal face, the second lumen is configured to be aligned with a camera at a distal face of the endoscope when the working channel of the endoscope is aligned with the first lumen.

D. The apparatus of C, wherein the distal end comprises a third lumen and a fourth lumen that extend from the hollow proximal end to the distal face, the third lumen and the fourth lumen are configured to be aligned with camera lights located at the distal face of the endoscope when the working channel of the endoscope is aligned with the first lumen.

E. The apparatus of any of A-C, wherein the tube comprises a stiffness value able to maintain the precurved configuration as the medical tool passes therethrough, such that as the medical tool exits the tube a longitudinal axis of the medical tool is at an acute angle relative to a longitudinal axis of the distal end of the endoscope.

F. An apparatus comprising: a base section comprising a first lumen extending from a proximal face to a distal face, wherein the first lumen comprises dimensions configured to receive a medical tool passed through a working channel of an endoscope, wherein the base section has an outer diameter based on an outer diameter of a distal end of the endoscope; and a tube extending from the distal face at the first lumen, wherein the tube has a precurved configuration configured to guide the medical tool received through the first lumen.

G. The apparatus of F, wherein at least one of the base section or the tube are visually transparent.

H. The apparatus of F or G, wherein the base section comprises a second lumen that extends from the proximal face to the distal face, the second lumen is configured to be aligned with a camera at a distal face of the endoscope when the working channel of the endoscope is aligned with the first lumen.

I. The apparatus of H, wherein the base section comprises a third lumen and a fourth lumen that extend from the proximal face to the distal face, the third lumen and the fourth lumen are configured to be aligned with camera lights located at the distal face of the endoscope when the working channel of the endoscope is aligned with the first lumen.

J. The apparatus of any of F-I, wherein the tube comprises a stiffness value able to maintain the precurved configuration as the medical tool passes therethrough.

K. A system comprising: an endoscope; and an endcap comprising: a hollow proximal end comprising an internal diameter configured to provide a frictional fit to a distal end of the endoscope; a distal end comprising a first lumen extending from the hollow proximal end to a distal face, wherein the first lumen comprises dimensions configured to receive a medical tool passed through a working channel of the endoscope; and a tube extending from the distal face of the endcap at the first lumen, wherein the tube has a precurved configuration configured to guide the medical tool received through the first lumen.

L. The system of K, wherein at least one of the hollow proximal end, the distal end of the endcap or the tube are transparent to a camera of the endoscope.

M. The system of K or L, wherein the distal end of the endcap comprises a second lumen that extends from the hollow proximal end to the distal face of the endcap, the second lumen is configured to be aligned with a camera at a distal face of the endoscope when the working channel of the endoscope is aligned with the first lumen.

N. The system of M, wherein the distal end of the endcap comprises a third lumen and a fourth lumen that extend from the hollow proximal end to the distal face of the endcap, the third lumen and the fourth lumen are configured to be aligned with lights located at the distal face of the endoscope when the working channel of the endoscope is aligned with the first lumen.

O. The system of any of K-N, wherein the tube comprises a stiffness value configured to maintain the precurved configuration as the medical tool passes therethrough.

While various embodiments of the disclosed technology have been described above, it should be understood that they have been presented by way of example only, and not of limitation. Likewise, the various diagrams may depict an example construction or other configuration for the disclosed technology, which is done to aid in understanding the features and functionality that can be included in the disclosed technology. The disclosed technology is not restricted to the illustrated example construction or configurations, but the desired features can be implemented using a variety of alternative construction and configurations. Indeed, it will be apparent to one of skill in the art how alternative functional, logical or physical partitioning and configurations can be implemented to implement the desired features of the technology disclosed herein. Also, a multitude of different constituent parts names other than those depicted herein can be applied to the various parts. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

What is claimed is:

1. An apparatus comprising:
a hollow proximal end comprising an internal diameter configured to provide a frictional fit to a distal end of an endoscope;
a distal end comprising a first lumen extending from the hollow proximal end to a distal face, wherein the first lumen comprises dimensions configured to receive a medical tool passed through a working channel of the endoscope; and
a tube extending from the distal face at the first lumen, wherein the tube has a precurved configuration configured to guide the medical tool received through the first lumen.

2. The apparatus of claim 1, wherein at least one of the hollow proximal end, the distal end or the tube are transparent to a camera of the endoscope.

3. The apparatus of claim 1, wherein the distal end comprises a second lumen that extends from the hollow proximal end to the distal face, the second lumen is configured to be aligned with a camera at a distal face of the endoscope when the working channel of the endoscope is aligned with the first lumen.

4. The apparatus of claim 3, wherein the distal end comprises a third lumen and a fourth lumen that extend from the hollow proximal end to the distal face, the third lumen and the fourth lumen are configured to be aligned with camera lights located at the distal face of the endoscope when the working channel of the endoscope is aligned with the first lumen.

5. The apparatus of claim 1, wherein the tube comprises a stiffness value able to maintain the precurved configuration as the medical tool passes therethrough.

6. An apparatus comprising:
a base section comprising a first lumen extending from a proximal face to a distal face, wherein the first lumen comprises dimensions configured to receive a medical tool passed through a working channel of an endoscope, wherein the base section has an outer diameter based on an outer diameter of a distal end of the endoscope; and
a tube extending from the distal face at the first lumen, wherein the tube has a precurved configuration configured to guide the medical tool received through the first lumen.

7. The apparatus of claim 6, wherein at least one of the base section or the tube are visually transparent.

8. The apparatus of claim 6, wherein the base section comprises a second lumen that extends from the proximal face to the distal face, the second lumen is configured to be aligned with a camera at a distal face of the endoscope when the working channel of the endoscope is aligned with the first lumen.

9. The apparatus of claim 8, wherein the base section comprises a third lumen and a fourth lumen that extend from the proximal face to the distal face, the third lumen and the fourth lumen are configured to be aligned with camera lights located at the distal face of the endoscope when the working channel of the endoscope is aligned with the first lumen.

10. The apparatus of claim 6, wherein the tube comprises a stiffness value able to maintain the precurved configuration as the medical tool passes therethrough.

11. A system comprising:
an endoscope; and
an endcap comprising:
a hollow proximal end comprising an internal diameter configured to provide a frictional fit to a distal end of the endoscope;
a distal end comprising a first lumen extending from the hollow proximal end to a distal face, wherein the first lumen comprises dimensions configured to receive a medical tool passed through a working channel of the endoscope; and
a tube extending from the distal face of the endcap at the first lumen, wherein the tube has a precurved configuration configured to guide the medical tool received through the first lumen.

12. The system of claim 11, wherein at least one of the hollow proximal end, the distal end of the endcap or the tube are transparent to a camera of the endoscope.

13. The system of claim 11, wherein the distal end of the endcap comprises a second lumen that extends from the hollow proximal end to the distal face of the endcap, the second lumen is configured to be aligned with a camera at a distal face of the endoscope when the working channel of the endoscope is aligned with the first lumen.

14. The system of claim 13, wherein the distal end of the endcap comprises a third lumen and a fourth lumen that extend from the hollow proximal end to the distal face of the endcap, the third lumen and the fourth lumen are configured to be aligned with lights located at the distal face of the endoscope when the working channel of the endoscope is aligned with the first lumen.

15. The system of claim 11, wherein the tube comprises a stiffness value configured to maintain the precurved configuration as the medical tool passes therethrough.
